(19) United States
(12) Patent Application Publication
(10) Pub. No.: US 2010/0094417 A1
(43) Pub. Date: Apr. 15, 2010

(54) Dual Hook Laryngeal Cable Prosthesis (DHLCP) used to correct airflow in the larynx of horses affected with left recurrent laryngeal neuropathy

(76) Inventors: Christopher Robert Johnson, Versialles, KY (US); James Schumacher, (US); Janet Ellen Morris, (US)

Correspondence Address: Sharon E. Loiselle, 1070 River Road, Weare, NH, 03281, US

(21) Appl. No.: 12/578,273

(22) Filed: Oct. 13, 2009

Related U.S. Application Data
(60) Provisional application No. 61/105,508, filed on Oct. 15, 2008.

(51) Int. Cl. A61F 2/20; A61D 99/00

(52) U.S. Cl. 623/9

(57) ABSTRACT

In a horse with left recurrent laryngeal neuropathy, abduction of the arytenoid cartilage on the affected side of the larynx cannot be achieved, causing progressive collapse of the arytenoid cartilage and vocal cord into the airway as negative inspiratory pressure increases. This produces airflow limitations that result in more rapid development of hypoxemia, hypercarbia, and metabolic acidosis, causing early fatigue and poor performance. Horses with left recurrent laryngeal neuropathy have a history of upper airway obstruction during exercise, including abnormal inspiratory respiratory noise and exercise intolerance. This noise is the result of air turbulence created as air passes over the affected vocal cord and ventricle, which acts as resonators. The DHLCP caudal and cranial device's are placed between the cricoid and the arytenoid cartilages and will permanently abduct the affected arytenoid cartilage. Once abducted sufficiently, it will provide unobstructed respiration during exercise, but not so much that saliva, food, and water are aspirated during swallowing.

### CROSS REFERENCE TO RELATED APPLICATION

This application claims priority of U.S. Provisional Patent Application No. 61/105,508, entitled “A Dual-Hook Laryngeal Cable Prosthesis (DHLCP) Used to Correct Airflow in the Larynx of Horses Affected with Recurrent Laryngeal Neuropathy”, which was filed on Oct. 15, 2008, and which is expressly incorporated herein by reference in its entirety

### BACKGROUND OF THE INVENTION

1. Field of the Invention

The present invention relates to a Dual-Hook Laryngeal Cable Prosthesis (DHLCP) used to correct airflow in the larynx of horses affected with left recurrent laryngeal neuropathy.

2. Description of the Related Art

U.S. Pat. No. 4,586,505 describes an example of another prior art device that may be referred to as a “laryngeal and tracheal stent”. A laryngeal and tracheal stent which can be used to reconstruct the larynx or trachea consists of an elongated cylindrical stenting cuff with an independently inflatable retention cuff at the top and a second independent inflatable retention cuff at the bottom. The stent also has individual cuff pilot tubes for separately inflating or aspirating the stenting, top and bottom cuffs, and guide means for moving the stent into place.

U.S. Pat. No. 4,794,924 describes an example of another prior art device that may be referred to as a “laryngeal stent”. A surgical device for insertion into a living being to support the larynx comprised of an elongated tubular member having a closed, crowed upper end. The device is comprised of a resilient biocompatible polymer material and includes an elongated, narrow aperture through the crowned upper end forming a normally closed valve-like arrangement.

U.S. Pat. No. 5,297,547 describes an example of another prior art device that may be referred to as a “laryngeal mask”. A laryngeal mask has an inflatable ring configured, upon inflation, to establish a peripheral seal around a patient's laryngeal inlet. Installation (insertion) is made in the fully deflated state, wherein the structural relation between the body of the mask and the inflatable ring is such that deflated ring surfaces become tightly opposed to each other so as to form a thin flange which peripherally surrounds the body of the mask and is concave on the posterior side of the mask; the concave flange effectively displaces all ring material away from the aperture of the mask, in the manner of the upturned brim of a hat. The concave flange is softly yieldable in its confinement by local body structures encountered in the course of mask insertion. And it is an important feature that the distal end of the deflated ring is adapted not only to smoothly ride posterior contours of the throat and pharynx but also to gently cam the epiglottis out of the path of insertial displacement of the mask while also assuring that the distal end of the deflated mask smoothly enters the upper sphinctral region of the esophagus. Once thus insertionally located, ring inflation will assuredly establish the desired peripheral seal of the mask around the laryngeal inlet.

U.S. Pat. No. 5,494,029 describes an example of another prior art device that may be referred to as a “laryngeal stent”. A laryngeal stent for supporting anatomical features of the mammalian larynx, including a human larynx is formed by a gas-inflatable envelope fabricated from a resilient, deformable material can conform to the anatomical surfaces of the larynx within the mammal's esophagus when pressurized by the gas. In preferred embodiment stents, the surface of the envelope is coarse, allowing for the venting of air between the laryngeal surfaces and the envelope.

However, the stent conforms so closely to the anatomical features of the larynx that sutures are not required to anchor the stent in place.

U.S. Pat. No. 5,623,921 describes an example of another prior art device that may be referred to as a “laryngeal mask airway and method”. A laryngeal mask airway

(LMA) is provided which can be easily and effectively used to intubate a patient with an endotracheal tube (ETT). The LMA of the invention is of the type having an inflatable mask and an airway tube connected to the mask. The airway tube comprises a first tube section having a distal end connected to the mask, and a second tube section having a distal end removably connected to the proximal end of the first tube section. The second tube section has longitudinally extending separation lines which allow it to be split apart. This feature enables complete removal of the LMA and successful incubation with the cuff of the ETT below the vocal cords of the patient.

U.S. Pat. No. 5,954,049 describes an example of another prior art device that may be referred to as a “equine mask”. A mask is provided for administering medication, particularly asthmatic medication, to equine animals, particularly horses. The mask has a body that fits over the nostrils of the horse. An aerosolization chamber is secured to the body and opens into the interior thereof, and is supplied with misted medications such as from a metered dose inhaler, For inhalation by the animal. The mask and body is also provided with one or more exhalation ports, and a strap or band or the like for securing the body to the animals face overlying the nostrils.

U.S. Pat. No. 5,983,897 describes an example of another prior art device that may be referred to as an “laryngeal mask assembly”. The laryngeal mask assembly has a mask portion formed by a mount with a plate to which inflatable cuffs are attached on both sides. The plate projects beyond the cuffs to form a non-inflatable leading tip to aid insertion of the assembly into the patient.

U.S. Pat. No. 6,070,581 describes an example of another prior art device that may be referred to as an “laryngeal airway device”. A laryngeal airway device includes an air tube with proximal and distal ends, and a sealing member attached to the distal end of the air tube. The sealing member includes an anterior surface adapted to seat in the throat, against the hyoid bone, and to stretch the laryngeal inlet. A hole extends through the anterior surface and the sealing member and communicates through an air passage with the distal end of the air tube. When the sealing member is seated in the throat, the hole is contained within the rim of the stretched laryngeal inlet, with which the anterior surface forms a seal that surrounds the hole. Airway patency is thereby ensured.

U.S. Pat. No. 6,119,695 describes an example of another prior art device that may be referred to as an “airway device”. The airway device for sealing against the laryngeal opening includes an air tube with proximal and distal ends and a sealing member attached to the distal end. The sealing member has a distal portion with a pair of opposing lateral flanges for engaging the cricoid cartilage to laterally align the sealing member with respect to the laryngeal inlet. The sealing member has a compressible anterior surface that contacts and seals against the laryngeal inlet. A tubular extension of the distal end of the air tube projects through and beyond the compressible anterior surface. The sealing member has a pronounced sigmoid shape having a lower section which, together with the tubular extension, creates a hook that provides an end point for accurate cephalad-caudad depth placement of the sealing member against the rim of the laryngeal inlet.

U.S. Pat. No. 6,830,049 B2 describes an example of another prior art device that may be referred to as a “laryngeal airway device”. The laryngeal airway device for sealing against the laryngeal opening includes an air tube with proximal and distal ends and a sealing member attached to the distal end. The airway device includes a tubular extension or snout for delivering air directly into the laryngeal opening; the snout is tapered and “hooded” in a manner that facilitates effective positioning of the airway device. The sealing member includes a coupler for coupling the device to an introducer. Complementing the laryngeal airway device is an introducer that includes a track for receiving the coupler of the laryngeal airway device and guiding the sealing member to a sealing position with respect to the laryngeal inlet.

U.S. Pat. No. 7,175,645 BI describes an example of another prior art device that may be referred to as a “nasal support device and method”. The nasal support device and method for facilitating air flow in the nasal passage of a domestic animal. The nasal support device (NSD) disclosed herein is useful for facilitating air flow during rest, physical exertion, respiratory ailment, etc. The NSD secures to the nose of a domestic animal to support the unsupported lateral vestibular walls of the nasal passages by lifting or stenting.

U.S. Patent Application Publication No. 2003/0037790 A1 describes an example of another prior art device that may be referred to as a “disposable laryngeal mask airway device”. The disclosed laryngeal mask airway device includes a mask portion and an airway tube. The mask portion includes an inflatable cuff, which defines a central opening when the cuff is inflated. The airway tube defines an internal passage extending from a proximal end of the tube to a distal end of the tube. The airway tube further defines two or more tabs disposed near the distal end of the tube. The mask portion is coupled to the airway tube near the distal end of the airway tube, and the tabs extend from the airway tube into the central opening defined by the cuff when the cuff is inflated.

U.S. Patent Application Publication No. 2003/0089375 A1 describes an example of another prior art device that may be referred to as a “breathing assistance improvements”. Assistance for breathing is provided for both humans and animals by providing for a member to adhere along only one or separately both sides of a nose and having a shape so that a part of the member can adhere over a bone of the nose and then in cantilever fashion adhere to and thereby support soft flesh forming an outer side of a nasal passage so as to keep this open even with heavy breathing.

U.S. Patent Application Publication No. 2005/0085753 A1 describes an example of another prior art device that may be referred to as a “throat support device and method”. A throat support device comprising a main throat support section having one or more lifting mechanisms, each with a tip, each tip adapted to fit into a location immediately adjacent to and behind a basihyoid bone, a thyroid cartilage and/or a cricoid cartilage of an animal to control upper airway dysfunction is disclosed. In one embodiment, the throat support device further comprises a bridle. In one embodiment, the upper airway dysfunction is dorsal displacement of the soft palate (DDSP) and the animal is a horse. Unlike conventional treatments involving surgery, the throat support device provides a non-invasive and convenient means of diagnosing and treating upper airway dysfunctions.

U.S. Patent Application Publication No. 2006/0027238 A1 describes an example of another prior art device that may be referred to as a “laryngeal-mask construction”. A laryngeal-mask construction comprises a tube body, provided with an external expanding portion at its one end, wherein said external expanding portion has a shape like a cap with indentations at its front sides and is provided with a stud on its front end, while on the lower other end of said tube body, an extending tube end with a circular plate is formed by extending therefrom; a hollow annular resilient support, which is also supported by a horse-shoe shape fenestrated plate bridging between the said hollow annular resilient support and the groove in the extending tube provided for joining around said external expanding portion; a pliant elastomeric beret, being sticky and tensile, and used for enclosing said external expanding portion and said hollow annular resilient support to form integrally a laryngeal-mask construction that, after inserting into the oral cavity of a patient, can make the distal airway opening closer to the larynx of the patient for achieving an effective seal and lessening the risk of aspiration.

Other examples of prior art devices include: U.S. Pat. Nos. 7,260,923 B2, 6,952,912 B2, 6,920,876 B2, 6,228,037 B1, 6,228,101 B1, 5,881,726, 5,460,125, 4,887,593, 4,273,119, 4,141,199, 4,044,531, 4,002,167, 3,952,335 and U.S. Patent Application Publications 2004/0231672 A1 and 2002/0056456 A1.

### BRIEF SUMMARY OF THE INVENTION

The present invention has been made considering the problems, and its object is to provide a dual-hook laryngeal cable prosthesis (DHLCP) and a method of instrumenting the dorsal aspect of a larynx of a horse to install the dual-hook laryngeal cable prosthesis (DHLCP) in order to correct airflow in the larynx.

Still other objects and advantages of the present invention will become readily apparent to those skilled in the art from the following detailed description wherein the embodiment is shown and described, simply by way of illustration of the best mode of the invention.

### BRIEF DESCRIPTION OF THE DRAWING FIGURES

FIG. 1 is a diagram of a dual-hook laryngeal cable prosthesis (DHLCP).

FIG. 2A is a diagram of a dual-hook in a dual-hook anchor arrangement.

FIG. 2B is a diagram of a shank in a dual-hook anchor arrangement.

### DETAILED DESCRIPTION OF THE PREFERRED EMBODIMENTS

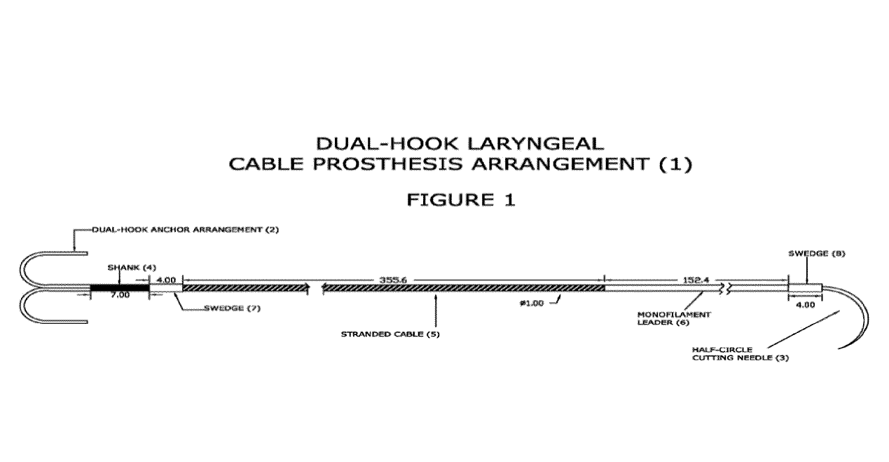

FIG. 1 illustrates an example of the dual-hook laryngeal cable prosthesis (DHLCP). The DHLCP (1) comprises a caudal device and a cranial device. Each of the devices comprises at one end a dual-hook anchor arrangement (2) and at the opposite end a half-circle cutting needle (3). The dual-hook anchor arrangement (2) and the half-circle cutting needle (3) are further connected by a shank (4), a stranded cable (5) and a monofilament leader (6). The dual-hook anchoring arrangement (2) is swedged to the stranded cable (5) by a first swedge (7). The first swedge (7) has a length of 4 mm and a diameter of 1.25 mm. The stranded cable (5) has a length of 355.6 mm and a diameter of 1 mm. The stranded cable (5) is further formed with a monofilament leader (6) at the end closest to the half-circle cutting needle (3). The monofilament leader (6) has a length of 152 mm and a diameter of 1 mm. The cranial device has a 26 mm half-circle cutting needle (3) swedged to the opposite end of the monofilament leader (6) by a second swedge (8). The caudal device has a 36 mm half-circle cutting needle (3) swedged to the opposite end of the monofilament leader (6) by a second swedge (8). The second swedge (8) has a length of 4 mm and a diameter of 1.25 mm.

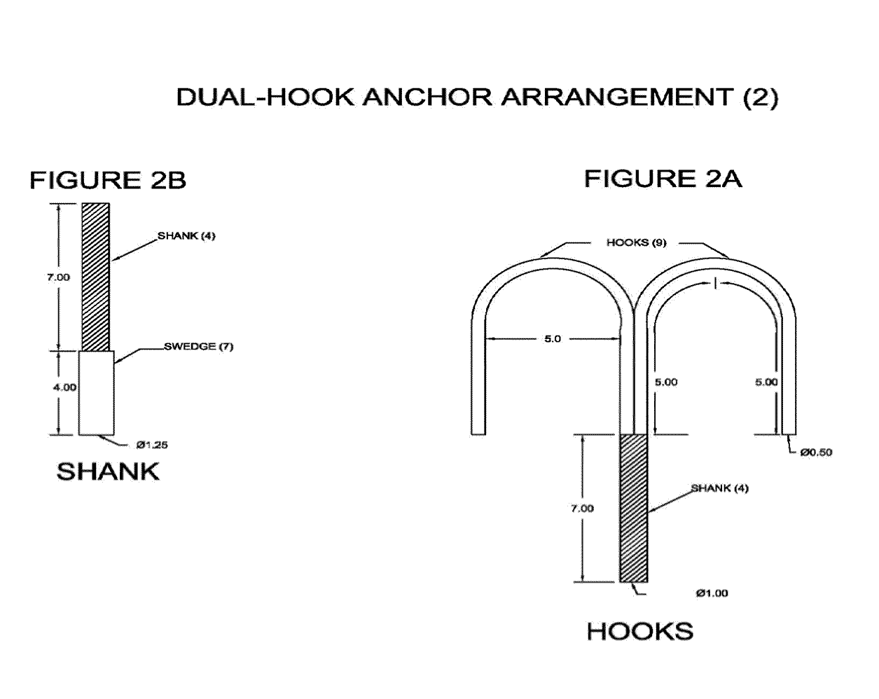

FIGS. 2A & 2B show specific details of a dual hook and shank for the dual-hook anchor arrangement (2) of FIG. 1. The dual-hook anchor arrangement (2) has two hooks (9) that connect to the shank (4) at approximately a 65 degree angle to each other. Each hook (9) has a length of 10 mm and a diameter of 0.5 mm. When connected to the shank (4), the bend of each hook (9) begins at 5 mm and turns upward and parallel to the shank (4). From the bend upward, the hook (9) has a length of 5 mm. The shank (4) has a length of 7 mm and a diameter of 1 mm.

The DHCLP (1) along with all of its components is made of implantable surgical stainless steel.

Commonly used surgical tools, a crimper, for example a Codman Crimper, a radiolucent cinch tightener, for example a Codman Radiolucent Cinch Tightener, and a cable cutter, for example a Codman Sof Wire Cable Cutter, are utilized when installing the DHCLP (1).

Next, a method of instrumenting the dorsal aspect of a larynx of a horse to install the dual-hook laryngeal cable prosthesis (DHLCP) in order to correct airflow in the larynx will be explained.

Using the left index finger as a guide, the half-circle cutting needle (3) of the caudal device is walked off the caudal aspect of the cricoid cartilage just lateral to the dorsal midline until the point slips beneath the cartilage. Next, the cutting needle (3) is advanced in a cranial direction, avoiding penetration of the point of the cutting needle (3) through the laryngeal mucosa. Next, the cutting needle (3) is rotated to penetrate the cricoid cartilage 1.5 cm to 2 cm cranial to its caudal border and lateral to the dorsal ridge. Next, the left index finger is used to ascertain the site of penetration. Next, the cutting needle (3) is drawn through the cartilage and out of an incision. The dual-hook anchor arrangement (2) should be seated onto the caudal edge of the cricoid cartilage with tension applied to the stranded cable (5) using the left hand. A large hemostat is passed beneath the cranial aspect of the cricopharyngeus muscle, and its jaws are brought out at the site of penetration of the cutting needle (3) in the cricoid cartilage. Next, the end of the stranded cable (5) is then grasped and drawn beneath the cricopharyngeus muscle and brought out of the incision. Next, after retracting the cricopharyngeus muscle caudally to expose the muscular process of the arytenoid cartilage, the cutting needle (3) of the cranial device is positioned on the caudomedial aspect of the muscular process and passed in a caudomedial to craniolateral direction through it. Next, the monofilament leader (6) and stranded cable (5) are pulled through the cartilage, and tension is placed on the stranded cable (5) to remove slack so that the dual-hook anchor arrangement is positioned tightly against the muscular process of the arytenoid cartilage. The stranded cable (5) is brought out of the incision. Next, after the cutting needles (3) are removed from the monofilament leaders (6), the leading ends of each stranded cable (5) from the caudal and cranial device are passed through a crimp in opposite directions. Next, a cinch tightener, for example a Codman Radiolucent Cinch Tightener, is used to apply tension on the stranded cables (5) until the desired abduction (observed endoscopically) of the arytenoid cartilage is reached, and a crimper, for example a Codman Crimper, is applied to the crimp to secure the stranded cables (5) together. The excess length of both stranded cables (5) is removed with cable cutter, for example a Codman Sof Wire Cable Cutter. The DHLCP (1) may be structured with or without a 3 mm barb back up each dual-hook (9) from the end point and flares 1 mm away to create the actual barb.

As will be realized, the invention is capable of other and different embodiments, and its several details are capable of modifications in various respects, all without departing from the invention. Accordingly, the drawings and description are to be regarded as illustrative in nature, and not in a restrictive or limiting sense, with the scope of the invention being indicated in the claims.

What is claimed is:

1. A Dual-Hook Laryngeal Cable Prosthesis (DHLCP) used to correct airflow in the larynx of horses affected with left recurrent laryngeal neuropathy comprising:
a. a caudal device; and
b. a cranial device.

2. The Dual-Hook Laryngeal Cable Prosthesis (DHLCP) used to correct airflow in the larynx of horses affected with left recurrent laryngeal neuropathy according to claim 1, wherein each of the caudal and cranial devices further comprises:
i. a dual-hook anchor arrangement (2);
ii. a shank (4);
iii. a first swedge (7);
iv. a stranded cable (5);
v. a monofilament leader (6);
vi. a second swedge (8);
vii. a half-circle cutting needle (3).

3. A Dual-Hook Laryngeal Cable Prosthesis (DHLCP) used to correct airflow in the larynx of horses affected with left recurrent laryngeal neuropathy according to claim 2, wherein the dual-hook anchor arrangement for each of the caudal and cranial devices further comprises:
a. two hooks (9) that connect to the shank (4) at approximately a 65 degree angle to each other; each hook (9) has a total length of 10 mm and a diameter of 0.5 mm; when connected to the shank (4), the bend of each hook (9) begins at 5 mm and turns upward and parallel to the shank (4);
and from the bend upward, the hook (9) has a length of 5 mm; the shank (4) has a length of 7 mm and a diameter of 1 mm; the dual-hook anchor arrangement (2) is swedged to the stranded cable (5) with the first swedge (7).

4. A Dual-Hook Laryngeal Cable Prosthesis (DHLCP) used to correct airflow in the larynx of horses affected with left recurrent laryngeal neuropathy according to claim 2, wherein the first swedge (7) for each of the caudal and cranial devices has a length of 4 mm and a diameter of 1.25 mm.

5. A Dual-Hook Laryngeal Cable Prosthesis (DHLCP) used to correct airflow in the larynx of horses affected with left recurrent laryngeal neuropathy according to claim 2, wherein the stranded cable (5) for each of the caudal and cranial devices has a length of 355.6 mm and a diameter of 1 mm.

6. A Dual-Hook Laryngeal Cable Prosthesis (DHLCP) used to correct airflow in the larynx of horses affected with left recurrent laryngeal neuropathy according to claim 2, wherein the monofilament leader (6) for each of the caudal and cranial devices has a length of 152.4 mm and a diameter of 1 mm.

7. A Dual-Hook Laryngeal Cable Prosthesis (DHLCP) used to correct airflow in the larynx of horses affected with left recurrent laryngeal neuropathy according to claim 2, wherein the caudal device has a 36 mm half-circle cutting needle (3) swedged to an end of the monofilament leader (6) by a second swedge (8).

8. A Dual-Hook Laryngeal Cable Prosthesis (DHLCP) used to correct airflow in the larynx of horses affected with left recurrent laryngeal neuropathy according to claim 2, wherein the cranial device has a 26 mm half-circle cutting needle (3) swedged to an end of the monofilament leader (6) by a second swedge (8).

9. A Dual-Hook Laryngeal Cable Prosthesis (DHLCP) used to correct airflow in the larynx of horses affected with left recurrent laryngeal neuropathy according to claim 7, wherein the second swedge (8) for each of the caudal and cranial devices has a length of 4 mm and a diameter of 1.25 mm.

10. A Dual-Hook Laryngeal Cable Prosthesis (DHLCP) used to correct airflow in the larynx of horses affected with left recurrent laryngeal neuropathy according to claim 8, wherein the second swedge (8) for each of the caudat and cranial devices has a length of 4 mm and a diameter of 1.25 mm.

11. A Dual-Hook Laryngeal Cable Prosthesis (DHLCP) used to correct airflow in the larynx of horses affected with left recurrent laryngeal neuropathy according to claim 2, wherein the DHLCP is made of surgical stainless steel.

12. A method of instrumenting the dorsal aspect of the larynx of a horse using a dual-hook laryngeal cable prosthesis (DHLCP), said method comprising the steps of:
a. walking off a half-circle cutting needle (3) from the caudal aspect of the cricoid cartilage just lateral to the dorsal midline until the point slips beneath the cartilage using the left index finger as a guide:
b. advancing the half-circle cutting needle (3) of the caudal device in a cranial direction and avoiding penetration of the point of the half-circle cutting needle (3) through the laryngeal mucosa;
c. rotating the half-circle cutting needle (3) of the caudal device to penetrate the cricoid cartilage 1.5 cm to 2 cm cranial to its caudal border and lateral to the dorsal ridge;
d. drawing the half-circle cutting needle (3) of the caudal device through the cricoid cartilage and out of an incision using the left index finger to ascertain the site of penetration;
e. seating the dual-hook anchor arrangement (2) of the caudal device onto the caudal edge of the cricoid cartilage and applying tension to the stranded cable (5) of the caudal device using the left hand;
f. passing a large hemostat beneath the cranial aspect of the cricopharyngeus muscle, and bringing out the hemostats jaws at the site of penetration of the half-circle cutting needle (3) of the caudal device in the cricoid cartilage;
g. grasping and drawing the end of the stranded cable (5) of the caudal device beneath the cricopharyngeus muscle and bringing the stranded cable (5) out of the incision;
h. positioning the half-circle cutting needle (3) of the cranial device on the caudomedial aspect of the muscular process of the arytenoid cartilage and passing in a caudomedial to craniolateral direction through the arytenoid cartilage, after retracting the cricopharyngeus muscle caudally to expose the muscular process of the arytenoid cartilage;
i. pulling the monofilament leader (6) and stranded cable (5) of the cranial device through the arytenoid cartilage;
j. positioning the dual-hook anchor arrangement (2) of the cranial device tightly against the muscular process of the arytenoid cartilage by removing slack and tensioning the stranded cable (5) and bringing the stranded cable (5) out of the incision;
k. removing the half-circle cutting needles (3) from both of the caudal and cranial devices from the monofilament leaders (6);
l. passing the leading ends of each of the stranded cables (5) from the caudal and cranial devices through a crimp in opposite directions;
m. applying tension from a cinch tightener on the stranded cables (5) of each of the caudal and cranial devices until the desired abduction (observed endoscopically) of the arytenoid cartilage is reached;
n. applying a crimper to a crimp to secure the stranded cables (5) of the caudal and cranial devices together, and removing any excess length of the stranded cables (5) from the caudal and cranial devices with a cable cutter.
